(19) United States
(12) Patent Application Publication
(10) Pub. No.: US 2003/0040114 A1
(43) Pub. Date: Feb. 27, 2003

(54) SMALL MOLECULE MODULATION OF RIBOZYMES

(75) Inventors: MEI CUI, ANN ARBOR, MI (US); ANTHONY WILLIAM CZARNIK, SAN DIEGO, CA (US); HOUNG-YAU MEI, ANN ARBOR, MI (US)

Correspondence Address: MERCHANT & GOULD PC, P.O. BOX 2903, MINNEAPOLIS, MN, 55402-0903, US

(73) Assignee: Warner-Lambert Company,, Marris Plains, NJ

(21) Appl. No.: 09/326,956

(22) Filed: Jun. 7, 1999

Related U.S. Application Data
(63) Continuation of application No. 08/923,487, filed on Sep. 4, 1997, now abandoned.
(60) Provisional application No. 60/024,685, filed on Sep. 5, 1996.

(51) Int. Cl. C12Q 1/68; C07H 21/04; C07H 21/02; C12N 15/85

(52) U.S. Cl. 435/455; 536/23.1; 435/6

(57) ABSTRACT

A method for selecting a compound that modulates the activity of a ribozyme in vivo in an organism comprising: (a) measuring in an assay the ability of a compound to selectively bind to a ribozyme thereby inhibiting the function of said ribozyme; and (b) selecting the assayed compound for use in modulating the activity of said ribozyme in vivo in an organism as a pharmaceutical agent as well as a method for selecting a compound for diagnosing the presence of a ribozyme in an organism that is pathogenic to an animal or plant.

### BACKGROUND OF THE INVENTION

The present invention relates to a method for identifying a compound that modulates the activity of a ribozyme in an organism. Additionally, the present invention relates to a method of selecting a compound for diagnosing the presence of a ribozyme in an organism that is pathogenic to an animal or plant. More particularly, the present invention relates to a screening procedure for selecting a compound for use in modulating the activity of a ribozyme in vivo in an organism as a pharmaceutical agent.

Nucleic acid molecules have been found to catalyze biochemical reactions in ways similar to protein enzymes. The initial findings by T. Cech and S. Altman, who subsequently shared the Nobel prize in Chemistry in 1989, focused on biochemical reactions of RNA substrates catalyzed by RNA molecules (ribozymes). Since their first discoveries, increasing examples of nucleic acid-catalyzed reactions that involve either RNA or non-RNA substrates have been discovered. A variety of natural or unnatural ribozymes and even catalytic DNA molecules have recently been reported to catalyze biochemical reactions.

Naturally occurring ribozymes have been found in various species. Among them, Group I intron RNA have been identified in microorganisms like bacteriophages, fungi, algae, protozoa, and eubacteria but not in higher eukaryotes (Cech, Annu. Rev. Biochem., 1990;59:543-68). With magnesium and guanosine as cofactors, Group I introns have been found to activate their own in vitro splicing in the absence of any proteins. A highly conserved secondary structure of all known Group I introns has been deduced from phylogenetic comparisons and biochemical analysis and suggested to be responsible for catalyzing the splicing reactions.

Since Group I introns and other classes of ribozymes, such as self-cleaving RNA in hepatitis delta virus, have been found in biologically relevant genes of several pathogenic microorganisms and each class involves specific mechanisms of biocatalysis that are likely not found in humans, it has been suggested that catalytic RNA could serve as a therapeutic target (von Ahsen, et al., Nature, 1991;353:368-70). Molecules that regulate, for example, the splicing of Group I intron-containing RNA are suggested to affect the growth of the microorganisms that contain these ribozymes (Liu, et al., Nucleic Acids Res., 1995;23:1284-91).

The present invention discloses two examples of human pathogens containing self-processing ribozymes. Pneumocystis carinii is an opportunistic fungus which causes fatal infection in patients with immunosuppressed systems. Although P. carinii pneumonia (PCP) is currently treated with pentamidine isethionate or a combination therapy of trimethoprim and sulfamethoxazole, neither treatment is always effective or safe. Furthermore, the mechanism of action for these drugs is not yet completely understood. It has been found that all small subunit rRNA genes in P. carinii include a self-splicing Group I intron. The Group I intron-catalyzed RNA splicing offers a unique target for therapeutic intervention of PCP. Hepatitis delta virus (HDV) represents another life-threatening human pathogen. Superinfection of hepatitis B virus-infected patients with HDV causes severe liver damage and death. Currently, high doses of interferon are the only effective treatment of HDV-infected patients. In the genome of HDV, a self-cleaving RNA has been found to be crucial for viral replication. Inhibition of this self-cleaving ribozyme system in HDV presents a potentially effective strategy in treating HDV infection.

Nucleic acid or amino acid-based compounds and metabolites such as streptomycin, pseudodisaccharides, and other aminoglycoside antibiotics (von Ahsen, et al., J. Mol. Biol., 1992;226:935-41) have been demonstrated to inhibit in vitro self-splicing of Group I introns. While these molecules represent the first examples of ribozyme inhibitors, there have been no reports of low molecular weight organic molecules that regulate the functions of self-splicing Group I introns. There is a need for methodologies, including high-throughput screening assays, for the rapid identification of low molecular weight organic modulators for ribozymes.

The present invention discloses methods that are amenable for automation or high-throughput screening to identify small molecule modulators for ribozymes. The present invention enables one to identify small organic molecules that regulate (activate or suppress) the activity of a particular ribozyme. The designed experimental conditions contain no other potential macromolecular targets (proteins, polysaccharides, or other nucleic acids). The present invention provides functional modulators specific for ribozymes without interference from other macromolecules. The present invention discloses preferred methods of labeling of nucleic acids and separation of starting material and final products. Preferred labeling methods include the use of radioactive isotopes such as ³²P, fluorescent tags such as fluorescein, or affinity tags such as biotin. Preferred separation methods include the use of biotin-streptavidin conjugation, nitrocellulose filtration, and gel electrophoresis. These methods are used to separate and quantitate the reactants and the products and to evaluate the effects of small molecules on the chemical reactions catalyzed by ribozymes of interest. The in vitro assays disclosed here include no proteins or other macromolecular targets other than ribozymes and the low molecular weight organic modulators thus identified are believed to interact directly with ribozyme molecules. These methods should be applicable to a variety of ribozyme or any DNA enzyme systems.

Most known ribozymes consist of specific sequences and/or structures and exhibit biological functions which are unlikely to occur in higher eukaryotes such as human. Ribozymes in an organism may be involved in a variety of activity including RNA splicing and RNA replication which are crucial for the organism to replicate. The organisms may be those microbials that infect animals or plant species. Small molecule modulators for a ribozyme should have therapeutic effect against the organism which utilizes this ribozyme for maintaining its regular life cycle. Due to the specific mechanism of action, modulators should have little effect on the hosts upon administration. In addition, if the existence is unique, a ribozyme could serve as a marker for certain pathogens. Modulators that bind specifically to this ribozyme and upon binding, provide detectable signals, could potentially be useful as a diagnostic tool in infectious diseases.

### SUMMARY OF THE INVENTION

Accordingly, a first aspect of the present invention is a method of selecting a compound that modulates the activity of a ribozyme in an organism comprising:

Step (a): measuring in an assay the ability of said compound to modulate the function of said ribozyme; and

Step (b): selecting the assayed compound for use in modulating the activity of said ribozyme in said organism.

A second aspect of the present invention is a method of selecting a compound that detects the presence of a ribozyme in an organism that is pathogenic for an animal or plant comprising:

Step (a): measuring in an assay the ability of a compound to selectively bind to said ribozyme;

Step (b): selecting the assayed compound for use in detecting said ribozyme in said organism; and

Step (c): utilizing said assayed compound in diagnosing the presence of said organism in said animal or plant.

### BRIEF DESCRIPTION OF THE DRAWINGS

The invention is further described by the following nonlimiting examples which refer to the accompanying FIGS. 1 to 11, short particulars of which are given below.

FIG. 1A shows the conserved secondary structure of Group I intron RNA.

FIG. 1B shows the sequence and secondary structure of Pneumocystis carinii Group I intron RNA.

FIG. 2 shows in vitro self-splicing of Pneumocystis carinii Ss-rRNA.

FIG. 3 shows an example of high-throughout screening data from the filtration assay. Number in each well represents the percentage of self-splicing reactions of the Pneumocystis carinii Group I intron RNA.

FIG. 4 shows a statistical analysis of the inhibition data from the filtration assay. Only 5% of all the tested samples inhibit ≧50% of the self-splicing reactions of the Pneumocystis carinii Group I intron RNA.

FIG. 5 shows an example of multiloading gel electrophoresis assay for self-splicing Pneumocystis carinii Group I intron RNA. On the same polyacrylamide gel, the same 20 samples were loaded at three different times. Within each round of loading, the order of loading for the 20 samples (from right to left) was different and is indicated below the data. Self-splicing reactions of Pneumocystis carinii Group I intron-containing RNA present two intensed RNA bands on the gel. Inhibition of the self-splicing reactions is indicated by the decreased intensity of these two bands. Inhibition in Samples 8, 10, 11, 12, 13, and 16 were observed regardless of when or where these samples were loaded.

FIG. 6 shows a correlation between the filtration and gel electrophoresis assays. Inhibition of self-splicing Pneumocystis carinii Group I intron RNA by 13 different compounds are shown.

FIG. 7 shows three inhibitors of self-splicing Pneumocystis carinii Group I intron RNA.

FIG. 8 shows the sequence and secondary structure of self-assembled ribozyme.

FIG. 9 shows RNA ligation catalyzed by a self-assembled ribozyme system

FIG. 10 shows an example of high-throughput screening data of the self-assembled ribozyme system. Number in each well represents the percentage of ligation reaction catalyzed by the self-assembled ribozyme.

FIG. 11 shows a self-cleaving hepatitis delta virus RNA.

### DETAILED DESCRIPTION OF THE INVENTION

In this invention, the term “small organic molecule” means a compound which has a molecular weight of less than about 1,000 daltons.

“Ribozyme” means an RNA molecule that catalyzes or enhances a biochemical reaction which involves the RNA molecule itself or other molecules.

“Modulation” means activation or suppression of the activity of a ribozyme.

“Detection” means the ability to detect the presence of a ribozyme in an organism contained in an animal or plant.

The present invention is based on the discovery that the functions of ribozymes can be modulated by the specific binding of small molecule modulators. The reported functions of ribozymes include, but are not limited to, the splicing reactions found in naturally occurring Group I introns and the functions of a ribonuclease, phosphotransferase, acid phosphatase, DNA and RNA restriction endonuclease, RNA ligase, RNA polymerase, and aminoacyl esterase. Regulation of ribozyme functions can be monitored by the separation of and the determination of the amount of the starting material and the end products. Preferred methods for separation include membrane filtration and gel electrophoresis. These methods have been used to study ribozymes alone or protein-RNA systems but have never been used in high-throughput screening for small organic molecule modulators of ribozymes. Examples are given to describe the present invention.

The following list contains abbreviations used within the specification:

| | |
|---|---|
| RNA | ribonucleic acid |
| DNA | deoxyribonucleic acid |
| rRNA | ribosomal ribonucleic acid |
| GTP | guanosine 5′-triphosphate |
| Tris-HCl | tris(hydroxymethyl)aminomethane-hydrochloride |
| (NH₄)₂SO₄ | ammonium sulfate |
| MgCl₂ | magnesium chloride |
| DMSO | dimethylsulfoxide |
| TCA | trichloroacetic acid |
| HCl | hydrogen chloride |
| Na₄P₂O₇ | sodium pyrophosphate |
| Mg²⁺ | divalent magnesium cation |
| NH₄Cl | ammonium chloride |
| KCl | potassium chloride |
| SPA | scintillation proximity assay |
| NaN₃ | sodium azide |
| HDV | hepatitis delta virus |
| nt | nucleotide |

The following nonlimiting examples illustrate the inventors' preferred method for carrying out the process of the present invention.

### EXAMPLE 1

### Screening Assay for Self-Splicing Group I Introns

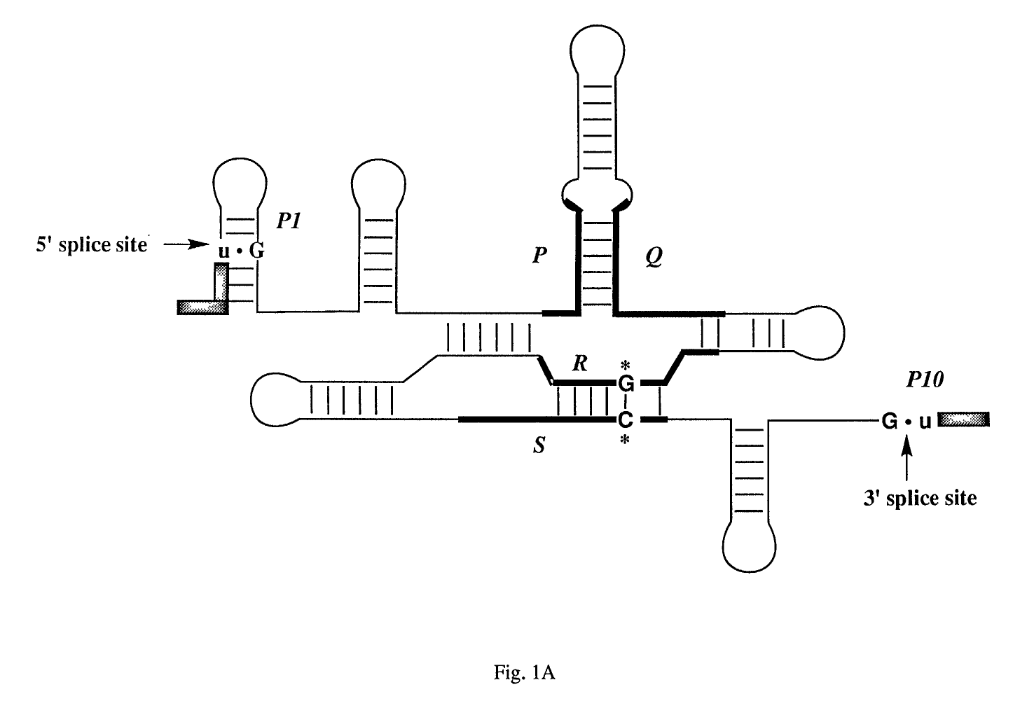

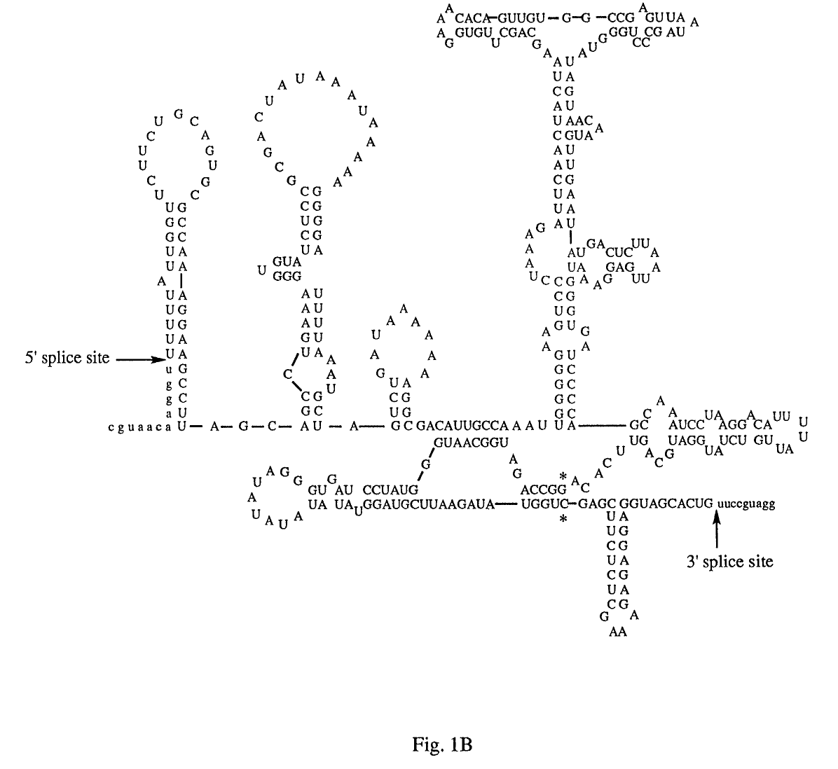

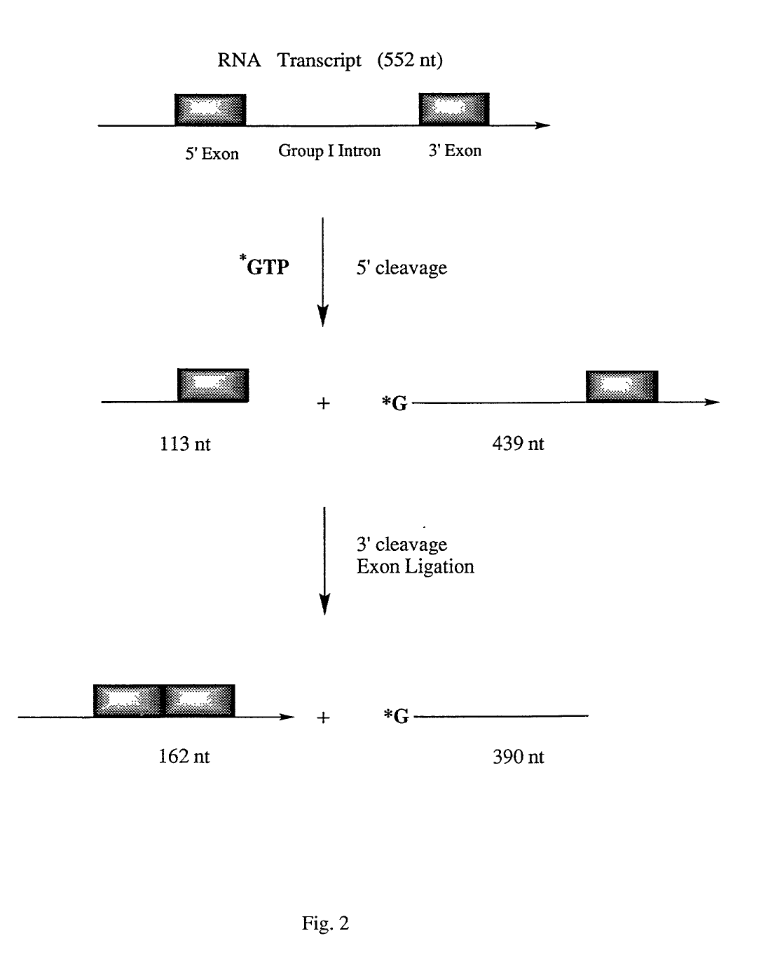

FIG. 1A shows the schematic representation of Group I intron RNA. The highly conserved secondary structure was deduced from phylogenetic analysis of more than 100 genomic sequences from various microorganisms. For proof of principle, self-splicing Group I intron RNA in Pneumocystis carinii have been used. In Pneumocystis carinii, all copies of chromosomal genes of small subunit ribosomal RNA have been found to contain these Group I introns (Lin, et ale, Gene, 1992;119:163-73). The splicing reactions are believed to be crucial in the maturation process of the biologically important Pneumocystis carinii rRNA. A reconstructed precursor RNA molecule (552-nucleotide long) containing the Group I intron of Pneumocystis carinii (390-nucleotide) and two short exon fragments is shown in FIG. 1B. FIG. 2 shows the two-step splicing reactions of this precursor RNA catalyzed by the cis-acting Group I intron in the presence of a cofactor (guanosine or 5′-phosphorylated guanosine) and divalent cations such as Mg²⁺. In this self-splicing process, the starting materials are full-length pre-rRNA (552-nt) and guanosine 5′-triphosphate (GTP). The products of the first cleavage reaction are intermediate RNA fragments; a 113-nt RNA containing the 5′-exon and a 439-nt RNA which consists of guanosine at the 5′-end, Group I intron, and the 3′-exon. The splicing reaction usually proceeds beyond the first step and the products from the second step include a ligated 5′- and 3′-exons (162-nt) and a released Group I intron (390-nt).

### Method A Filtration Assay

A high-throughput screening assay for self-splicing Group I introns using 96-well format filter plates is disclosed. Nonradioactive, intron-containing precursor RNA is incubated with α-³²P-labeled GTP in the absence or presence of small molecules. During the first step of Group I intron splicing, ³²P-GTP is covalently ligated to the 5′ terminus of the intron. Subsequently, the intron-containing products of both the first (439-nt RNA) and the second (390-nt RNA) steps are radioactively labeled at the 5′-end with ³²P isotopes. Free ³²P-GTP and the longer ³²P-labeled (acid-precipitable) RNA products can be readily separated through trichloroacetic acid precipitation and subsequent filtration through nitrocellulose membrane. The efficiency of inhibition can be followed by measuring the amount of the radioactivity retained on the filter membrane. In the control experiment, the spliced products containing 5′-³²P GMP will retain on the membrane. If the first step (5′-cleavage) is inhibited by a small molecule, incorporation of ³²P-GTP to the precursor RNA will be inhibited and, therefore, a decreased amount of radioactivity will be found on the membrane.

In each well of a 96-well U-bottom microtiter plate, 32 μL of precursor RNA (in self-splicing buffer 50 mM Tris-HCl, pH 7.5, 10 mM (NH₄)₂SO₄, 10 mM MgCl₂, 5 mM spermidine, and 5% glycerol) was added with 2 μL of a small organic compound in dimethylsulfoxide (DMSO) and the mixture incubated at room temperature for 5 minutes. This incubation ensures pre-equilibration between the enzymes and the inhibitors before addition of a 6 μL solution of α-³²P-GTP (≈10,000 cpm in self-splicing buffer) to initiate the self-splicing reaction. The reaction mixture was incubated at 50° C. for 3 hours before 150 μL of 11% trichloroacetic acid (TCA) was added to stop the splicing and to precipitate RNA. The TCA/reaction mixture was incubated at room temperature for 5 minutes and then transfered to a nitrocellulose filter plate (Millipore, MHAB, pre-treated with 100 μL of 0.05% polyethylenimine for 15 minutes). Filtration was performed on a vacuum manifold (Millipore, MAVM) and the filter membrane was washed once with 200 μL of washing buffer (0.1N HCl, 100 mM Na₄P₂O₇). The filter was allowed to dry and the retained radioactivities were determined using scintillation counting (Wallac Microbeta Counter). Due to the use of the 96-well microtiter plates, all solution handling was automated by using a robotic workstation (Beckman, Biomek 1000).

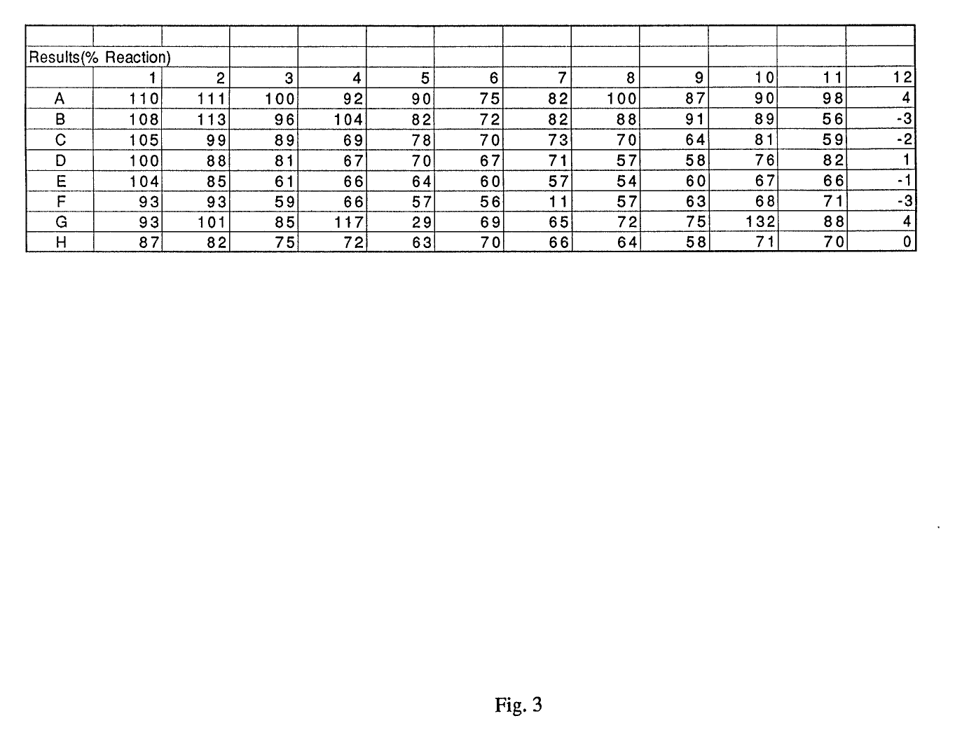

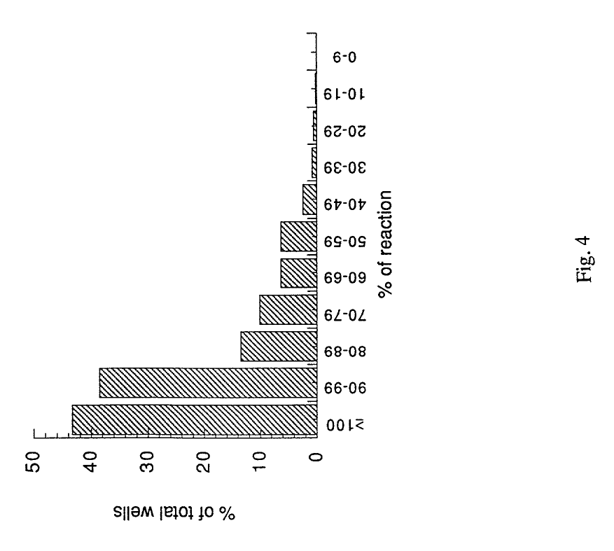

A typical example of the results obtained from this high-throughput assay is shown in FIG. 3. Column 1 represents the results of eight repeats of the self-splicing reactions in the absence of any inhibitors while Column 12 represents the data of eight repeats of solution containing ³²P-GTP only. Other control experiments such as self-splicing in the absence of Mg²⁺ or self-splicing reaction at time zero showed similar data as those of ³²P-GTP only. The difference between the mean values of Columns 1 and 12 serves as the common denominator in calculating the inhibitory effect. The percentage in each well was obtained by subtracting the means of Column 12 from raw data in each well and then dividing this value by the common denominator. Each percentage value represents the remaining of the self-splicing reaction. Eighty different samples of potential inhibitors were tested per plate. The eight repeats in Column 1 (or Column 12) suggest that there may be up to 20% error associated with this filter assay. This should not affect the usage of this filter method as a primary screening assay if the high-throughput screen is to quickly identify significant positive or negative effects in a large collection of compounds. The results of a total of 18 plates (a total of 1,440 samples) have been analyzed and the statistical distribution of the inhibitory activities is shown in FIG. 4. This analysis is useful to optimize the screening conditions and to determine the selection of inhibitors from high-throughput screening.

### Method B Gel Electrophoresis Assay

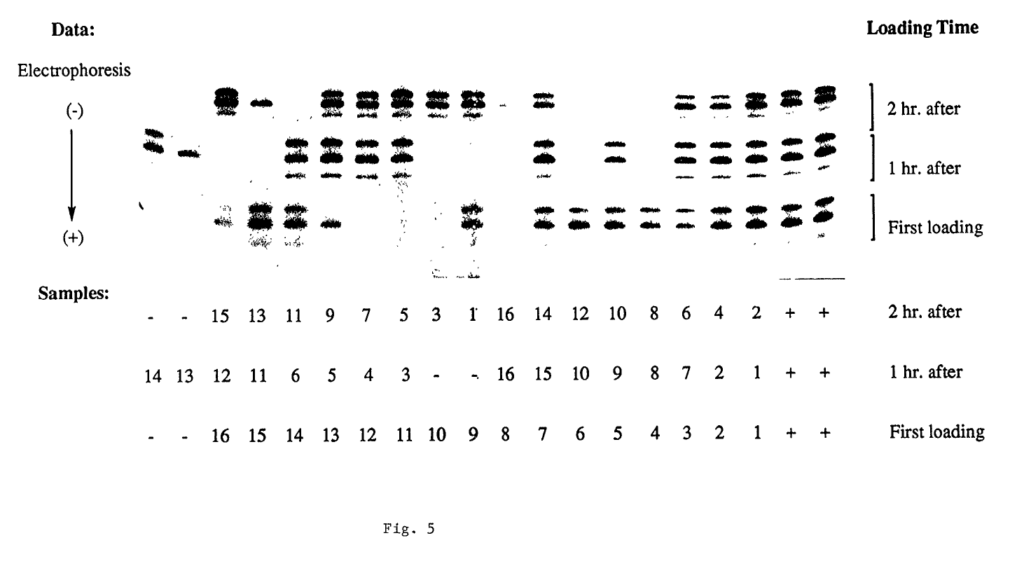

As another example of a separation method useful in the present invention, a high-throughput gel electrophoresis assay with multiple loading capability is used in studies of self-splicing Group I introns. To be specific, the reaction sample, prepared similarly as described in the filtration assay, was loaded on a denaturing polyacrylamide gel (7 M urea, 6% polyacrylamide) and electrophoresed for 1 hour at room temperature. Due to their lengths, the ³²P-labeled products of the self-splicing introns can be readily distinguished from the ³²P-GTP on a polyacrylamide gel. On a single polyacrylamide gel, usually three or four repetitive loadings of different samples into the same wells at discrete times present no interference between samples. FIG. 5 shows the image of an 6% polyacrylamide gel (dimension 15×15 cm) on which 20 different samples were loaded onto a single well at time 0, 1, and 2 hours after the gel electrophoresis began. After a total of 3 hours of electrophoresis, the desired products are well-resolved in all samples regardless of the time of loading.

FIG. 5 shows the autoradiograph of a typical polyacrylamide gel containing three separate loadings of a set of 16 samples. Each sample contains the same self-splicing reaction solution as described in the previous section (32 μL of precursor RNA in self-splicing buffer, 2 μL of DMSO or compound, and 6 μL solution of α-³²P-GTP). As described previously, the products of the self-splicing reactions are 5′-end labeled intron-containing 439-nt RNA (from the first step) and 390-nt RNA (from the second step). These large RNA fragments can be readily separated from the starting material, ³²P-GTP. After splicing reactions, a total of 20 samples were loaded on a polyacrylamide gel three times (first loading at time 0, then two more loadings at 1, and 2 hours after the first loading, respectively). To verify the validity of this multiple loading technique, at each loading procedure, the samples were loaded with different orders. As shown in FIG. 5, the first loading follows the sequence (from right to left) of samples that contain self-splicing reactions with no compounds added (two lanes, labeled as “+”), self-splicing in the presence of Compound 1, 2, . . . , 16, (16 lanes, labeled as 1, 2, . . . , 16), and two repeats of control samples containing ³²P-GTP only (two lanes, labeled as “−”). At the second and third loadings, the order of samples loaded on the same gel were as described in the FIG. 5. As shown in FIG. 5, samples containing Compounds 8, 10, 11, 12, 13, and 16 can be readily identified as inhibitors (“hits”) regardless of when and where they were loaded. In this gel electrophoresis assay, for the purpose of quantitation of reaction yields, appropriate ³²P-labeled RNA fragments can be introduced to each sample as an internal standard immediately prior to loading. The gels containing self-splicing products and internal standards separated according to their different mobilities can be dried and quantitated by a phosphor imager (Phosphoimager, Molecular Dynamics). Although more laborious than the filtration assay, the gel electrophoresis method described here has been demonstrated to be useful in high-throughput screening with Group I intron ribozyme as a molecular target.

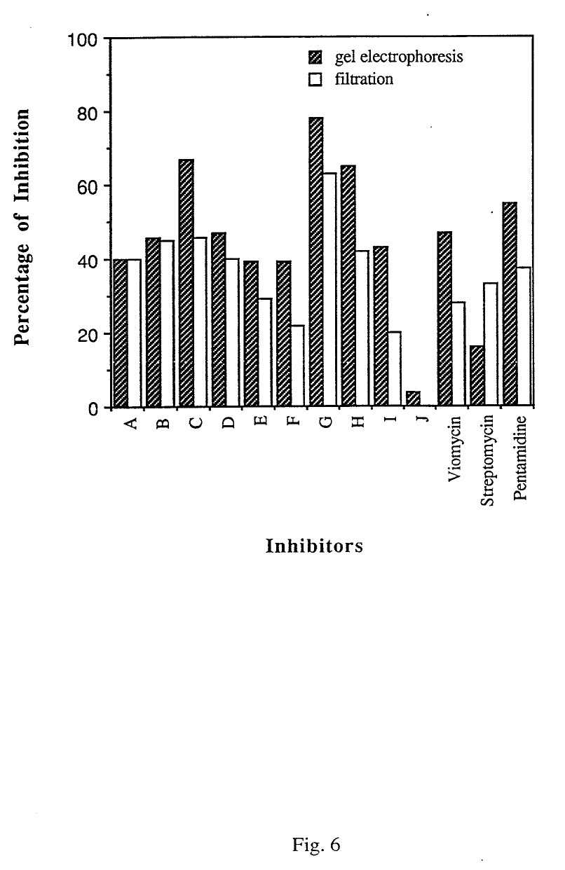

In general, there is a good correlation between the filtration and the gel electrophoresis assays. The most active (or inactive) compounds can be readily identified by the filtration assay and verified by the gel electrophoresis method. The self-splicing inhibitory effects of 12 compounds were examined using both the filtration assay and the gel electrophoresis method. In each sample, a single concentration (20 μM) of an inhibitor was prepared and aliquots were removed for either filtration or gel electrophoresis. Four repeats of each sample were performed and the average inhibition of each inhibitor was presented in FIG. 6. Viomycin, streptomycin, and pentamidine, previously reported as Group I intron inhibitors at high μM concentrations, were also tested in both assays as controls. For examples, Compounds C and G are found to be the most active inhibitors in both filtration and gel electrophoresis assays. On the other hand, Compound J was found to be inactive in both assays. Under similar conditions, viomycin, streptomycin, or pentamidine exhibited either low or modest activity.

In both the filtration or gel electrophoresis assays, compounds that modulate (either up- or down-regulate) the splicing reactions can be readily identified by comparing the relative amount of products versus starting materials in the control sample, which contains no compounds. For example, in the filtration assay (FIG. 3), compared with wells containing control samples (Column 1), a decrease of almost 90% radioactivity in Well F7 indicates strong inhibition of self-splicing reactions in the presence of compounds contained in that particular well.

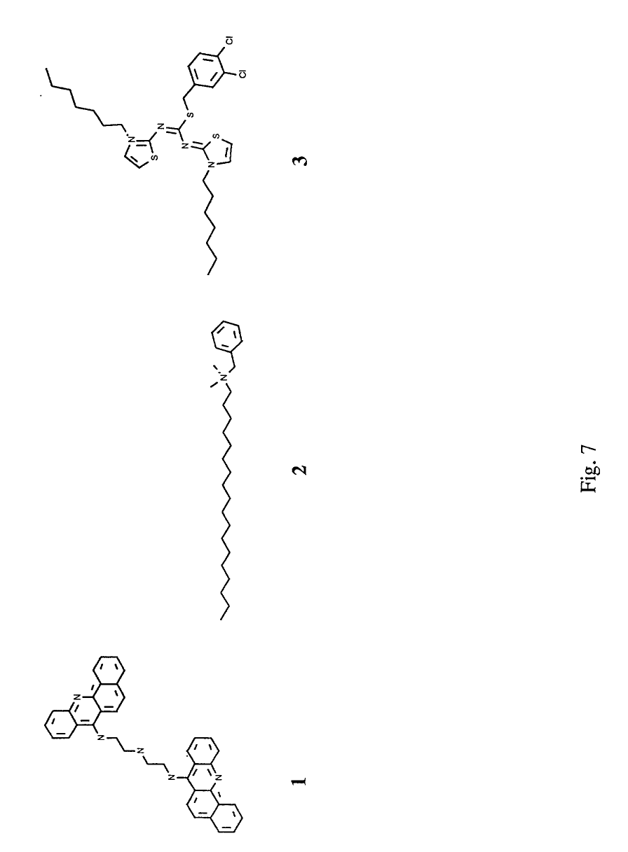

A compound library of approximate 150,000 members has been investigated using the present method and some identified inhibitors for the self-splicing reactions of P. carinii Group I intron are provided here. Compounds 1, 2, and 3 (shown in FIG. 7) represent three distinct inhibitors for self-splicing Group I intron. These compounds were initially identified as active inhibitors from high-throughput screening using the present filtration assay and further verified with the gel electrophoresis assay described above. These compounds present their inhibitory effects as a function of compound concentration with IC₅₀ values around 5 μM. Although the mechanism of inhibition may vary, these examples indicate that structurally different modulators for self-splicing Group I intron RNA can be identified from a compound library using the present methods.

### EXAMPLE 2

### Modulation of the Functions of a Self-assembled Ribozyme

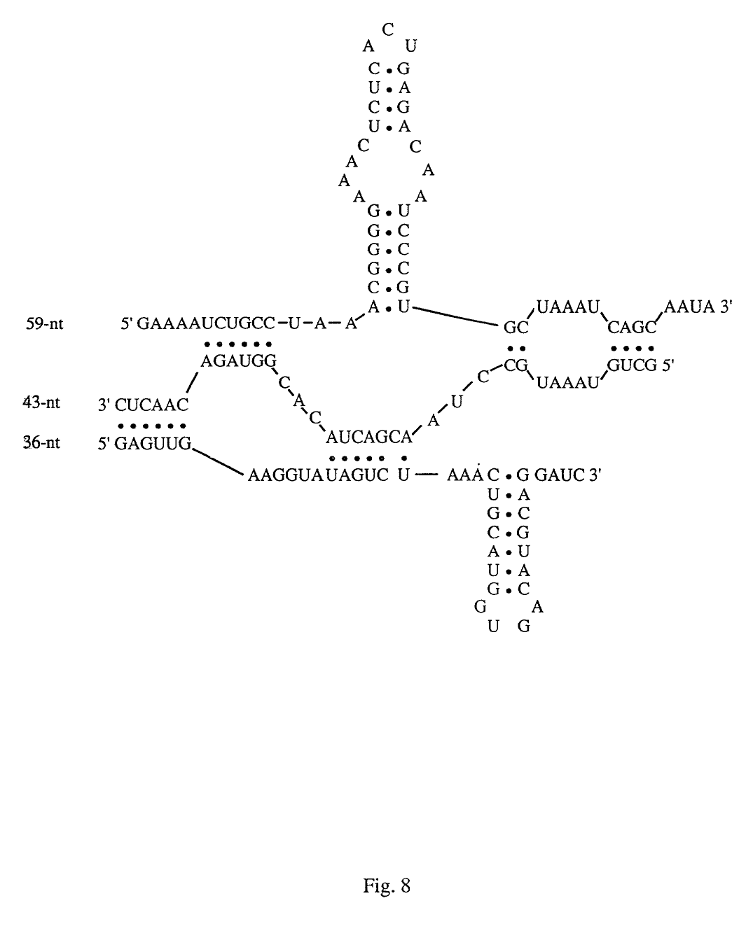

As shown in FIG. 1A, the well-defined catalytic core of Group I intron includes conserved sequences P, Q, R, and S, a G/C base pair as the binding site for guanosine, and the P1 and P10 segments containing the 5′ and 3′ splice sites, respectively. As described earlier, there has been an increasing number of chemical reactions found to be catalyzed by Group I intron RNA. The activities found for this ribozyme include that of a ribonuclease, phosphotransferase, acid phosphatase, DNA and RNA restriction endonuclease, RNA ligase, RNA polymerase, and aminoacyl esterase. Most interestingly, all these reactions use the same active site as the splicing reaction. Recently, RNA fragments containing P, Q, R, and S sequences have been demonstrated to assemble to form a multisubunit ribozyme that catalyzes an RNA ligation reaction (Doudna, et al., Science, 1991;256:1605-8). As shown in FIG. 8, this self-assembled ribozyme system is composed of three RNA fragments of 59-nt, 43-nt, and 36-nt, respectively. It has been demonstrated that this ribozyme, in the presence of Mg²⁺, catalyzed the ligation of a 6-nt RNA fragment to a 28-nt RNA, shown in FIG. 8. The assembled ribozyme catalyzes the nucleophilic attack of the 3′-OH from the 6-nt to the 3′-phosphodiester linkage following the 5′-guanine residue of the 28-nt. The products include a 3′-hydroxy-guanosine from the 5′-end of the 28-nt RNA substrate and a ligated 33-nt RNA product. This ligation reaction represents a mimicry of either the reversal of the first step or the second step reaction that occurred in Group I intron RNA self-splicing. Regulation of the self-assembled ribozyme system may serve as a model for regulating any biological reactions catalyzed by the Group I intron RNA.

### Method A Filtration Assay

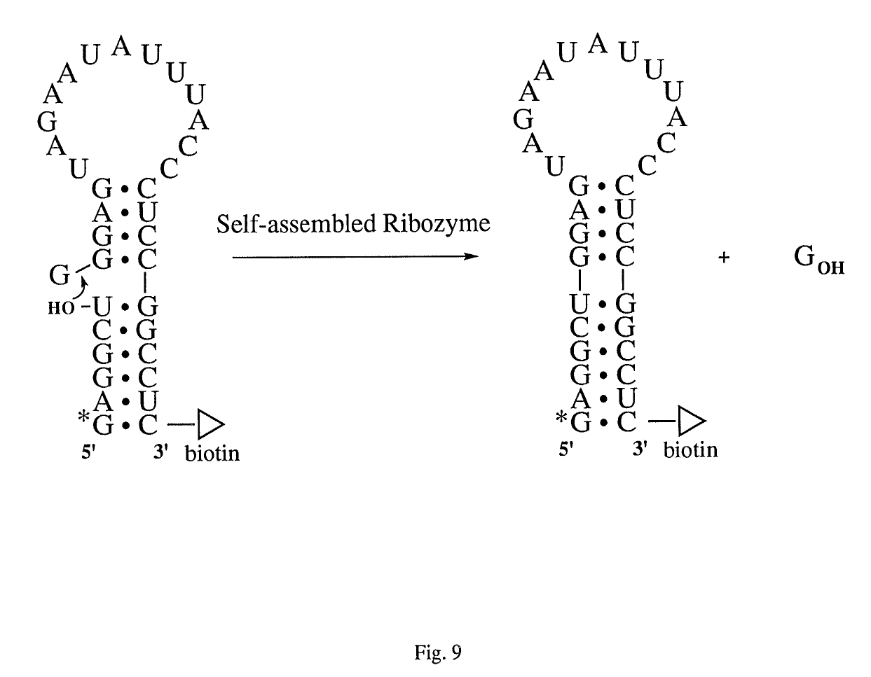

A high-throughput filtration assay capable of screening compounds that regulate the RNA ligation reaction catalyzed by the self-assembled RNA (ribozyme) system has been established. The present method includes the use of radioisotope (e.g., ³²P) labeling at the 5′-end of the 6-nt RNA substrate and the incorporation of a biotin molecule at the 3′-end of the 28-nt RNA substrate. The ligation reaction catalyzed by the self-assembled ribozyme system shown in FIG. 9 should generate a 5′-³²P, 3′-biotinylated 33-nt RNA product which can be readily distinguished from all the other RNA components in the mixture. To facilitate the separation of the 33-nt product from the 6-nt RNA, a protocol of biotin-streptavidin conjugation is incorporated into this assay. To be specific, the 6-nt RNA is 5′-labeled with ³²P and the 28-nt RNA is 3′-tagged with a biotin molecule. When ligation reactions occur, the 33-nt product should be 5′-³²P-labeled and 3′-biotin-tagged and, upon conjugation with streptavidin, can be readily separated from the ³²P-labeled 6-nt RNA. Since it is not radioactively labeled, the 28-nt substrate RNA does not interfere with the detection of the product formation.

The assay is performed in 96-well format microtiter plates described as follows. In each well, 48 μL of the three-fragment ribozyme solution (in 30 mM Tris-HCl, pH 7.5; 150 mM MgCl₂; 10 mM NH₄Cl; 400 mM KCl; 10% polyethylene glycol, annealed at 55° C. for 10 minutes then cooled down to 37° C. gradually) is added to 3 μL of compounds in dimethyl sulfoxide (final concentration of 20 μM) The mixture is then equilibrated for 5 minutes at room temperature and added to 9 μL of substrate RNA solution. The final concentration of the ribozyme is 50 nM, of the 28-nt substrate is 20 nM, and of the 6-nt substrate is ≈100 pM (≈10,000 cpm). The reaction is incubated at 37° C. for 2 hours and then added to 180 μL of 8 M urea/formamide solution (2:1 volume ratio). The addition of urea/formamide solution significantly reduced the amount of nonspecific binding of unligated RNA substrates to streptavidin. After 5 minutes of mixing and standing, an aliquot (120 μL) of the mixture is transfered to a well of a 96-well filtration plate (Millipore, MHAB) which is pre-wetted with cold washing buffer (e.g., 10 mM phosphate, pH 7.2, and 150 mM NaCl, 0.05% NaN₃, and 5% glycerol). In each well, the urea/reaction mixture is then added with 100 μL of streptavidin-coated SPA beads (Amersham International, 0.625 mg/mL in washing buffer). After equilibrating for 10 more minutes, filtration is performed using a Multiscreen Vacuum Manifold (Millipore). The filters are washed with cold washing buffer once and dried before determining their radioactivities using a Wallac Microbeta Liquid Scintillation Counter.

The relative amounts of the ³²P-labeled 6-nt and 33-nt product can be determined by the radioactivity retained on the filter membrane. The 33-nt biotinylated product conjugated with streptavidin-coated SPA beads will retain on the filter while free 6-nt and unbound compounds will pass through during filtration. The biotinylated 28-nt RNA substrate itself is not radiolabeled and, therefore, does not interfere with the product analysis on the filter. Compounds that regulate (either up- or down-regulate) the designed ligation reactions will be identified based on the differential radioactivity retained on the filter compared with the control sample which contains no compounds

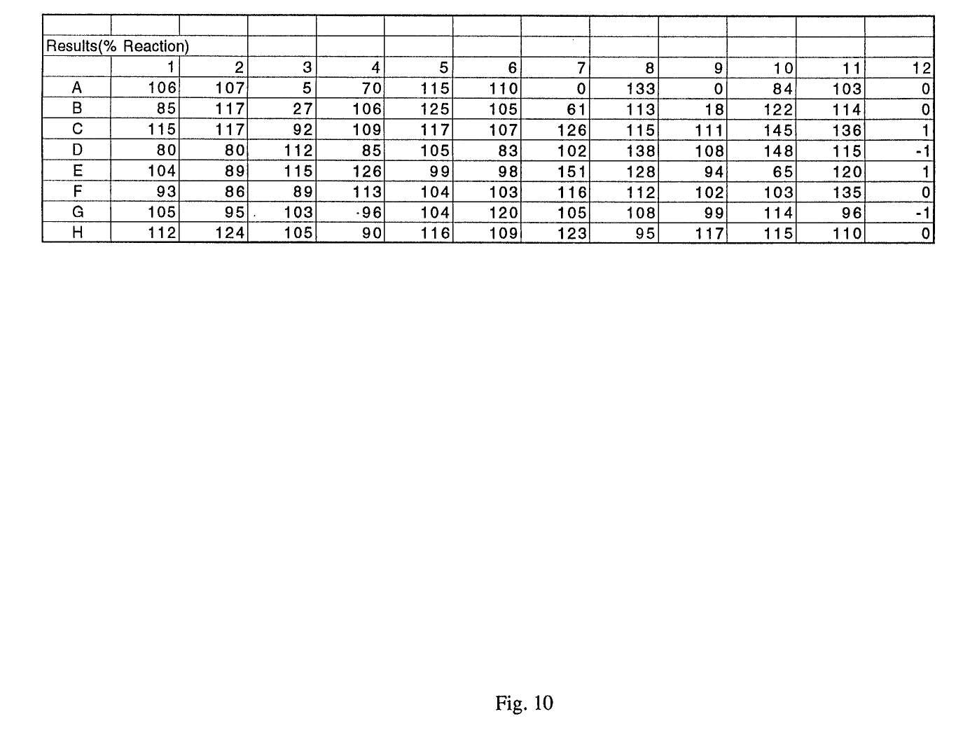

The results obtained from this high-throughput assay in a 96-well microtiter plate are shown in FIG. 10. Column 1 represents the results of eight repeats of the ligation reactions catalyzed by the self-assembled ribozyme in the absence of any inhibitors while Column 12 represents the data of eight repeats of solution containing RNA substrates (³²P-6-nt and 28-nt) only. Other control experiments, such as ribozyme-catalyzed ligation reaction at time zero, showed similar data as those of RNA substrates only. The difference between the mean values of Columns 1 and 12 serves as the common denominator in calculating the regulatory effect. The percentage in each well was obtained by subtracting the means of Column 12 from raw data in each well and then dividing this value by the common denominator. Each value represents the percentage of the ligation reaction in the presence of added compounds. Eighty different samples of potential inhibitors or activators were tested per plate. The eight repeats in Column 1 (or Column 12) suggest that there may be up to 20% error associated with this filter assay. This should not affect the usage of this filter method as a primary screening assay if the high-throughput screen is to quickly identify significant positive or negative effects in a large collection of compounds. As shown in FIG. 9, compared to control samples, decreased radioactivity found in certain wells indicates ligation is inhibited by the presence of compounds. In theory, compounds that up-regulate the catalysis can also be identified by the increased counts in radioactivity.

### Method B Scintillation Proximity Assay

An alternative way of detection is the use of scintillation proximity assay. In the ligation experiment described above, the final product, 33-nt ligated RNA, is biotinylated and can be immobilized on the SPA beads through biotin-streptavidin conjugation. If the RNA is labeled with β particle-emitting isotopes such as ³³P, ³⁵S, and ³H, the efficiency of ribozyme-catalyzed ligation can be followed by the light emitted from the scintillant embedded in the SPA beads. This detection method eliminates the separation procedure and simplifies significantly the procedure of a screening assay.

### EXAMPLE 3

### Modulating the Activity of the Self-cleaving RNA in Hepatitis Delta Virus

Chronic hepatitis D is a severe and rapidly progressive liver disease. Hepatitis D virus (HDV) is the infectious agent of delta hepatitis. Replication of HDV requires helper functions, e.g., provision of its envelope, from hepatitis B virus (HBV). HDV is a unique, small circular single-stranded RNA (1.7 kilobases) with extensive self complementarity that allows the RNA to fold into an unbranched rod-like structure. The replication of this circular RNA (plus strand) is believed to undergo a rolling-circle mechanism, which leads to formation of a multimeric-length RNA intermediate. Site-specific cleavage of the multimeric intermediate produces a monomer (minus strand) that is subsequently circularized through an as yet unknown self-ligation mechanism. The self-cleavage activity of HDV has been suggested to be essential for viral replication in cells. Single-base mutations known to affect in vitro self-cleavage reaction exert effects in HDV RNA replication in both hepatic and nonhepatic cell lines. It is therefore expected that inhibition of the self-cleavage reaction of HDV should prevent the viral replication and subsequently the progression of this chronic disease. Because the target RNA sequence and the self-cleaving function are unique to the pathogen, high selectivity is expected for human therapeutics.

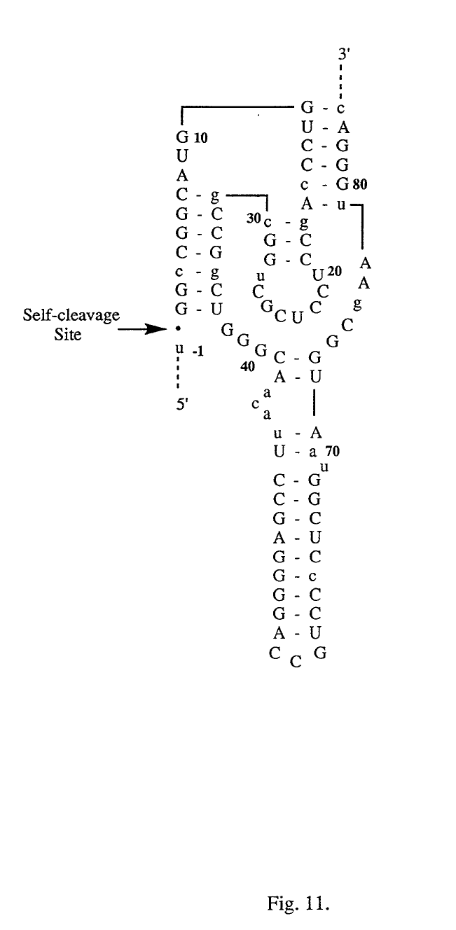

A high-throughput in vitro assay to identify small molecules that interfere with the self-cleavage reaction of HDV RNA is needed. The minimum length of the HDV RNA that undergoes the self-cleavage reaction is 86 nucleotide long RNA (shown in FIG. 11). The cleavage reaction requires only millimolar amounts of divalent cation and proceeds at neutral pH and ambient or body temperatures. The cleavage occurs specifically at the first 5′-nucleotide (between u•G), resulting in a shorter (e.g., single nucleotide) and a much longer (e.g., 85-nt) RNA fragment. Appropriate procedures such as trichloroacetic acid precipitation and membrane filtration described in previous examples will be used to separate RNA fragments that are different in length. An 86-nt HDV RNA that is labeled with ³²P isotope at the 5′-end will be used. The efficiency of inhibition can be followed by measuring the amount of the radioactivity retained on the filter membrane. In the control experiment, 5-³²P-labeled HDV RNA self-cleaves and produces a ³²P-labeled nucleotide which passes through the membrane and an unlabeled (and undetectable in this assay) 85-nt RNA fragment that will retain on the membrane. When the self-cleavage is blocked by inhibitors, however, an increased amount of full-length HDV RNA and, therefore, an increased amount of radioactivity will be found on the membrane.

Since there are no proteins involved in the autocatalytic process of HDV, small molecules that inhibit the self-cleavage reaction are most likely binders of HDV RNA. The hits identified from this assay can then be evaluated for their activities in cellular or in vivo models. From the proposed HDV screen, it is reasonable to suspect that we can find small molecules that will either up- or down-regulate the self-cleavage reactions of HDV RNA. In the HBV/HDV coinfected patients, small molecules that can attenuate the replication of HDV should have the potential to subsequently regulate the infection caused by HBV.

The previous examples describe a reproducible, sensitive, and high-throughput assay for discovering modulators of the self-splicing reactions of Group I intron. Current studies were focused on the intron sequence obtained from P. carinii, but the modulators identified in this assay should have the potential to block similar self-splicing reaction in other Group I intron systems. Thus, modulators acting on this specific mechanism could be of clinical utility in treating infections caused by microorganisms whose life cycle is regulated by the catalytic function of Group I introns. If such agents are shown to be clinically useful, then in vitro assays described here might be more generally used to screen agents targeting a variety of RNA-catalyzed reactions. Similarly, modulators for other ribozymes including but not limited to the two other examples provided in this invention or for DNA enzymes may be useful in regulating any biological systems which contain these enzymes. The present methods can also distinguish modulators based on their capability of enhancing or suppressing the catalytic properties of the ribozymes.

The present invention discloses methods which allow rapid determination of small organic modulators that regulate the functions of ribozymes, for example, the in vitro self-splicing process of Group I introns and the self-cleavage activity of HDV RNA. The modulators identified by the present methods are then subjected to further studies including specificity, toxicity, cellular, and in vivo activity. For specificity testing, the activity of modulators is examined in the presence of excess amount of other nucleic acids, such as calf thymus DNA or transfer RNA, which do not affect the properties of the target ribozyme. This process should eliminate nonspecific nucleic acid effectors such as certain intercalators. The ribozyme-specific modulators are then submitted to certain cell lines for cytotoxicity testing at the doses where the modulators exhibit their efficacies. Modulators that are specific for a ribozyme and exhibit acceptable cytotoxicity are then administered to cellular cultures of a microorganism which contains the ribozyme. The therapeutic activity of the selected modulators are then investigated in in vivo studies with animals or plants which are infected by the microorganisms.

In addition, high-throughput screening methods disclosed in the present invention not only identify small molecule modulators of ribozymes but also specific binding agents for various ribozymes. If changes in properties such as spectroscopic or biophysical characteristics of these agents (or their analogues) are associated with their binding to ribozymes, these agents can be used for diagnosis of the existence of certain ribozyme sequences in biological systems of interest.

1. A method of selecting a compound that modulates the activity of a ribozyme in an organism comprising:
Step (a): measuring in an assay the ability of said compound to modulate the function of said ribozyme; and
Step (b): selecting the assayed compound for use in modulating the activity of said ribozyme in said organism.

2. A method according to claim 1 wherein the compound is a small organic molecule.

3. A method according to claim 2 wherein the small organic molecule has a molecular weight of less than 1,000 daltons.

4. A method according to claim 1 wherein the ribozyme is an RNA molecule that catalyzes or enhances a biochemical reaction which involves the RNA molecule or other molecules.

5. A method according to claim 4 wherein the ribozyme has a motif selected from the group consisting of: a Group I intron; and a hepatitis delta virus.

6. A method according to claim 1 wherein the activity of a ribozyme is either activated or suppressed.

7. A method according to claim 1 wherein functionally the activity of a ribozyme is selected from the group consisting of: a ribonuclease; a phosphotransferase; an acid phosphatase; a restriction endonuclease; an RNA ligase; an RNA polymerase; and an aminoacyl esterase.

8. A method according to claim 1 wherein the organism is a pathogen that infects an animal or plant.

9. A method according to claim 1 wherein in Step (a) the assay is a biological assay.

10. A method according to claim 9 wherein the biological assay is selective for a compound that modulates a specific ribozyme without interference from other macromolecules.

11. A method according to claim 10 wherein the biological assay is a high-throughput screen.

12. A method according to claim 11 wherein the biological assay is selected from the group consisting of: a filtration assay; a gel electrophoresis assay; and a scintillation proximity assay.

13. A method according to claim 1 wherein the selected compound is useful in treating an infection caused by a microorganism containing said ribozyme.

14. A method according to claim 13 wherein the selected compound is useful in treating Pneumocystis carinii infections.

15. A method according to claim 1 wherein the selected compound is useful in treating delta hepatitis virus infections.

16. A method according to claim 1 wherein the selected compound is useful in treating chronic hepatitis D.

17. A method of selecting a compound that detects the presence of a ribozyme in an organism that is pathogenic for an animal or plant comprising:
Step (a): measuring in an assay the ability of a compound to selectively bind to said ribozyme;
Step (b): selecting the assayed compound for use in detecting said ribozyme in said organism; and
Step (c): utilizing said assayed compound in diagnosing the presence of said organism in said animal or plant.

18. A method according to claim 17 wherein the compound is a small organic molecule.

19. A method according to claim 18 wherein the small organic molecule has a molecular weight of less than 1,000 daltons.

20. A method according to claim 17 wherein the ribozyme is an RNA molecule that catalyzes or enhances a biochemical reaction which involves the RNA molecule or other molecules.

21. A method according to claim 20 wherein the ribozyme has a motif selected from the group consisting of: a Group I intron; and a hepatitis delta virus.

22. A method according to claim 17 wherein in Step (a) the assay is a biological assay.

23. A method according to claim 22 wherein the biological assay is selective for a compound that modulates a specific ribozyme without interference from other macromolecules.

24. A method according to claim 23 wherein the biological assay is a high-throughput screen.

25. A method according to claim 24 wherein the biological assay is selected from the group consisting of: a filtration assay; a gel electrophoresis assay; and a scintillation proximity assay.

26. A method according to claim 17 wherein the selected compound is useful in diagnosing an infection caused by a microorganism containing said ribozyme.

27. A method according to claim 26 wherein the selected compound is useful in diagnosing Pneumocystis carinii infections.

28. A method according to claim 17 wherein the selected compound is useful in diagnosing delta hepatitis virus infections.

29. A method according to claim 17 wherein the selected compound is useful in diagnosing chronic hepatitis D.
